(19) United States
(12) Patent Application Publication
(10) Pub. No.: US 2012/0150001 A1
(43) Pub. Date: Jun. 14, 2012

(54) Hyaluronic acid based glucose monitoring

(75) Inventors: Walter Jeffrey Shakespeare, Macungie, PA (US); Walter Gregory Shakespeare, Macungie, PA (US)

(73) Assignee: COMPACT IMAGING

(21) Appl. No.: 13/373,055

(22) Filed: Nov. 3, 2011

Related U.S. Application Data
(60) Provisional application No. 61/456,285, filed on Nov. 4, 2010.

(51) Int. Cl. A61B 5/1455

(52) U.S. Cl. 600/316; 600/310

(57) ABSTRACT

The invention provides a method for using OCT human tissue scan data for tracking a scan structure in depth, follow change in structure position within an OCT scan from, for example, fasting glucose level to peak glucose level and back down again, and relate the structure position change to analyte concentration. In the preferred embodiment, the analyte of interest is glucose concentration and the target of interest is living human skin. A hyaluronic acid based mechanism is suggested for dermis thickness. Alternate embodiments of the method are presented, including curve fitting of topographic regions corresponding to trackable target features.

### RELATED APPLICATIONS

This application claims priority from U.S. provisional 61/456,285, filed Nov. 4, 2010, by the same inventors, the entirety of which is incorporated by reference as if fully set forth herein.

### GOVERNMENT FUNDING

None

### FIELD OF USE

The invention relates to application of interferometric techniques, such as OCT, for bio-medical measurements of tissue including in vivo measurements. The invention further relates to measurements for the purpose of tissue characterization and analysis.

### BACKGROUND

Glucose concentration in humans may be measured non-invasively using optical coherence tomography (OCT). Specificity of noninvasive blood glucose sensing using optical coherence tomography technique: a pilot study, Kirill V Larin, Massoud Motamedi, Taras V Ashitkov and Rinat O Esenaliev, Phys. Med. Biol. 48 (2003) 1371-1390.

OCT typically uses a super-luminescent diode (SLD) as the optical source, as described in Proceedings of SPIE, Vol. 4263, pages 83-90 (2001). The SLD output beam has a broad bandwidth and short coherence length.

The OCT technique involves splitting the output beam into a probe and reference beam. The probe beam is applied to the system to be analyzed (the target). It is understood that to apply a probe beam means to move the beam over a specific patch of skin of predetermined area in such a way as to average the scans so that speckle (coherent interference, i.e. constructive and destructive optical interference) is minimized while the physical topography in depth of the averaged area is retained in the scan.

Light scattered back from the target is combined with the reference beam to form the measurement signal. Because of the short coherence length only light that is scattered from a depth within the target such that the total optical path lengths of the probe and reference are equal combine interferometrically. Thus the interferometric signal provides a measurement of the scattering value at a particular depth within the target. By varying the length of the reference path length, a measurement of the scattering values at various depths can be measured and thus the scattering value as a function of depth can be measured.

However, a number of difficulties still exist in quickly and accurately using OCT to determine changes in glucose in, for example, a diabetic human. One difficulty is associating scans obtained by OCT to meaningful representations of glucose concentration.

At a gross level, differences in the skin tissue structures can profoundly alter the shape and characteristic of an OCT scan. Hair follicles, surface blemishes, sweat glands, and changes in skin structures that occur along with change is glucose concentration all contribute to difficulties in interpreting OCT scan data.

Changes that may occur within the skin itself (i.e. skin substance and structures) along with changes in glucose concentration in the skin have been observed and reported. [INSTITUTE OF PHYSICS PUBLISHING PHYSICS IN MEDICINE AND BIOLOGY, Phys. Med. Biol. 48 (2003) 1371-1390 PII: S0031-9155(03)58795-4, Specificity of noninvasive blood glucose sensing using optical coherence tomography technique: a pilot study, Kirill V Larin1,2, Massoud Motamedi2, Taras V Ashitkov1,2 and Rinat O Esenaliev1,2,3,4] In particular, it has been observed and reported that food intake affects circulating levels of hyaluronan (also referred to as hyaluronic acid) in humans. [J. R. E. Fraser & P. R. Gibson, “Mechanisms by which food intake elevates circulating levels of hyaluronan in humans,” Journal of Internal Medicine 2005: 258: 460-466.]

It is known that hyaluronan is largely present in the dermis layer of the skin. It is also known that hyaluronan characteristically binds many times its molecular weight in water molecules. The cosmetic industry has used hyaluronan in products designed to provide an appearance of youthful fullness to aging skin that has thinned as a consequence of age-related changes.

What is needed is a method of tracking changes in skin between fasting and peak glucose, and to correlate skin thickness change to glucose concentration. What is also needed is a method of characterizing changes in the topography of tissues, and in particular, of skin. In other words, a method of using OCT scans of tissue to track glucose by measuring the change in thickness of the skin between fasting and peak glucose levels (or simple two different glucose levels), and a method of correlating the change is skin thickness to glucose concentration is a need not currently met. What is also needed is a means to characterize changes in tissue topography that correspond to changes in analyte levels, where the analyte of interest may be, for example, glucose.

### BRIEF SUMMARY OF THE INVENTION

The invention herein meets at least all of the aforementioned unmet needs. The invention provides a method for using OCT scan data for tracking a structure in depth, following changes in structure position in OCT scan and scan topography from, for example, fasting glucose level to peak glucose level and back down again, and relating such changes in OCT scan to changes in analyte concentration. In the preferred embodiment, the analyte of interest is glucose concentration and the target of interest is living human skin.

### BRIEF DESCRIPTION OF THE DRAWINGS

The following drawings, as well as figures within the Appendix hereto, are useful in the understanding of the invention:

FIG. 1 depicts generalized OCT system elements, a tissue target, and an OCT scan, which may be an average of multiple scans of the same location, with scan portions corresponding to skin structures marked.

FIG. 2 depicts human skin tissue.

FIG. 3 depicts OCT generated scan results of skin tissue.

FIG. 3 shows OCT generated scan results of skin tissue, showing selected scan features and the shift effect according to the inventive method.

FIG. 4 is a flow chart illustrating the method according to a preferred embodiment.

### DETAILED DESCRIPTION OF A PREFERRED EMBODIMENT

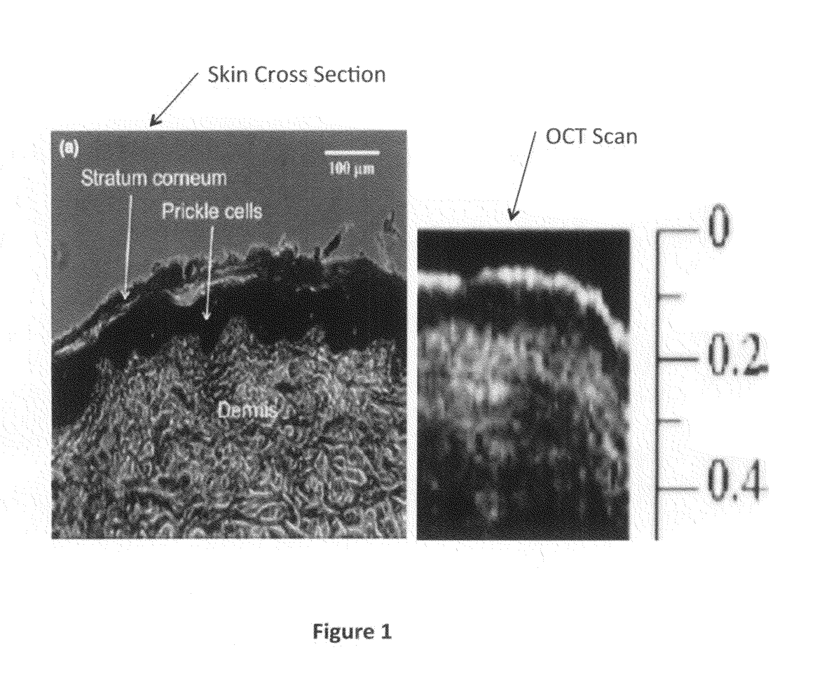

The method according to the preferred embodiment employs OCT System scan data of tissue targets as depicted in FIG. 1. FIG. 1 depicts generalized OCT system elements, a tissue target, and an OCT scan with scan portions interpreted as corresponding to skin structures marked. OCT is well known, and the illustration is provided as an aid to the reader to visualize how the depth information obtained from the OCT appears in a scan of the target The depicted OCT system elements are a broadband light source 101, a beam splitter 103, a reference mirror 105 and a detector 107. Light from the source that penetrates the target 109, passing from the skin surface 111, to some depth in the target, scattering, and from which light scattered within the target, a portion will be reflected back to the beam splitter 103, and then to the detector 107.

As can be seen by referring to the OCT scan 113 of the target 109, characteristics of the scan 113 can be attributed to depth characteristics of the target. The illustrative OCT acquired scan 113, shows epidermis (stratus corneum), prickle cell layer, and dermis. For OCT images of the various subsurface layers, and discussions of the scattering characteristics observed, we refer the reader to Alex et. al, Multispectral in vivo three-dimensional optical coherence tomography of human skin, Journal of Biomedical Optics, vol. 15(2), March April 2010.

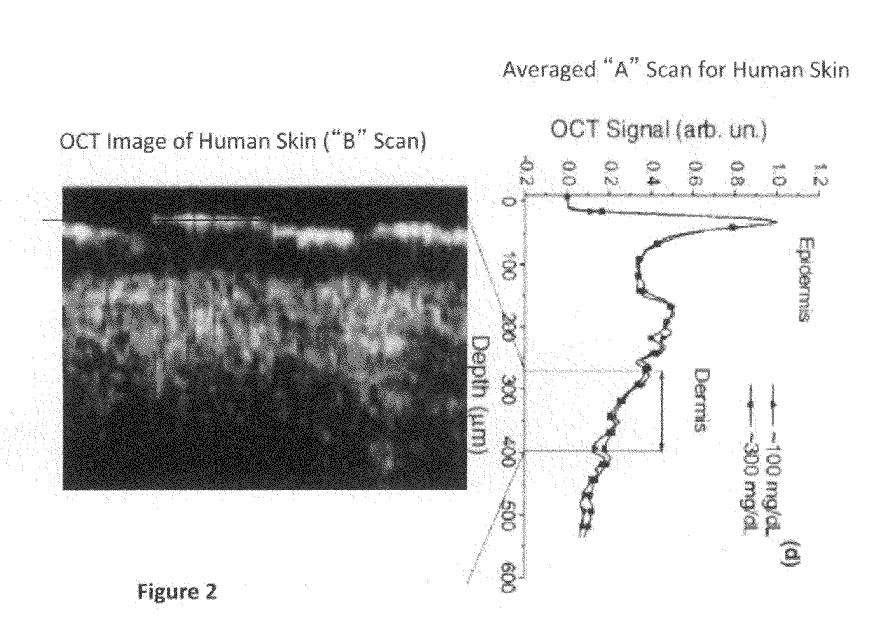

FIG. 2, taken from FIG. 1 of Alex et al: Multispectral in vivo three-dimensional optical coherence tomography of human skin, Journal of Biomedical Optics, March/April 2010, vol 15(2), illustrates human skin tissue in greater detail. As can be seen by referring to FIG. 2, and to FIGS. 8a and 8b in the Appendix hereto, the outermost region is the epidermis EPI 201, with the region beneath the epidermis comprised of a papillary dermis PD 203 region and a reticular dermis RD 205. In the recticular dermis, capillary and subcutaneous fat may also be seen.

As a further aid to understanding, please refer to Appendix A, and especially FIGS. 8a and 8b for more additional detailed description. In particular note how specific dermal features such as blood vessels, sweat glands, hair follicles and collagen show as specific topography features (mountains and valleys) in the OCT depth scan. Structures in the skin are localized, and, depending on scan area, some portion of such a localized skin structure will appear as a scan feature in the A scan, and will add character to a topographic reading. A helpful additional reference on skin composition may be found on-line at the website for the University for Western Australia.

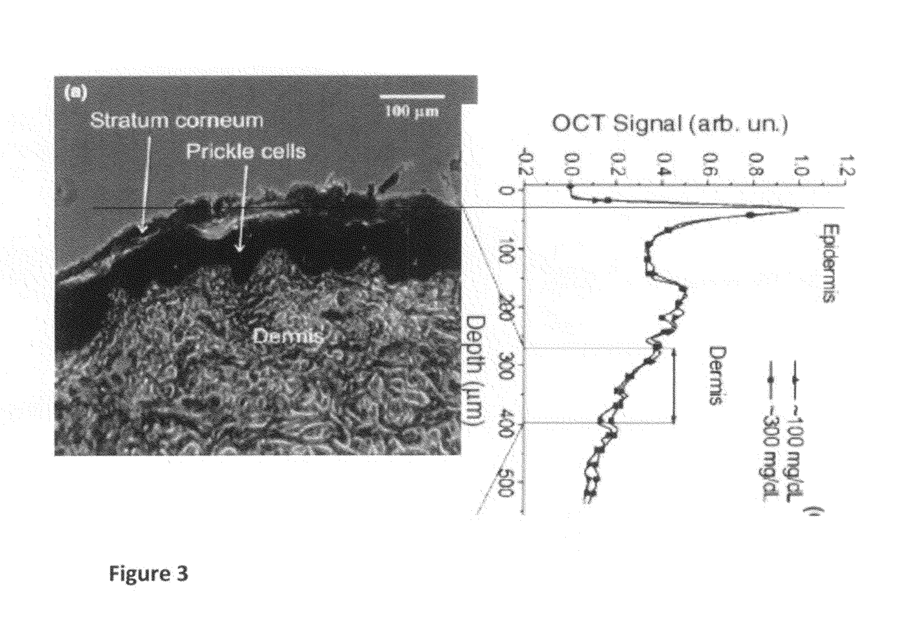

FIG. 3 is a generalized depiction of two OCT scans, S1 and S2, and illustrates the application of the inventive method to analyzing scans of human tissue. In the preferred embodiment, S1 and S2 would consist of scans taken at fasting and peak glucose levels, respectively. The measurement D1 represents the distance between two selected peaks of S2, and D2 represents the distance between corresponding peaks in S1. D3 represents the distance from the midpoint of the peak representing the epidermal layer (see, for example, FIG. 1) to the midpoint of a first selected peak in S2, and D4, the distance to the corresponding selected peak in S1. The method provides for selecting features of a first scan S1, identifying the corresponding features in a second scan S2, and analyzing the differences. Differences amenable to useful analysis include peak height, depth, shape and structure, especially as related to identifying the same structures from the two different scans, as well as changes in relation to other scan features.

As has already been mentioned in the discussion of FIG. 2 hereinabove, topographic reading of the first and second scan can reveal changes in skin structure shape and depth. A further depiction of changes in skin topography, including depth signature, may be seen in FIG. 5 in the Appendix.

In the preferred embodiment, the change in position of selected features in Scan 1 and Scan 2, where the timing of each scan is selected to correspond to different levels of glucose, it has been observed that the magnitude of the change is approximately a linear function of depth depending on the specific structural elements within the scanned area, such as blood vessels, sweat glands, hair follicles, etc. These elements may not have the same hyaluronan content as the surrounding ground substance in the dermis. As used herein, “position of selected features” is intended to include any trackable scan feature or set of features within the topography of an OCT scan.

It can be appreciated that the selected scan features may be any of a wide variety of tissue structures, such as a cell layer, that result in scan characteristics. Similarly, the analysis is not limited to peaks, but may include troughs, or any number of other identifiable scan topographies of greater or lesser complexity.

In the preferred embodiment, the shift in peak features is attributed to change in the thickness of the dermis from fasting to peak glucose levels. It is the conclusion of the inventors that the amount of feature shift bears a reproducible and useful relationship to the change in concentration of glucose. A lengthier discussion is set forth in the Appendix A, including further exposition of evidence supporting the presence of hyaluronan in human dermis, as well as mechanism related to changes in dermis thickness and tissue topography.

One additional point with respect to the shape or topography changes is that if the mechanism were strictly dermal bulk index of refraction change due to glucose, the shapes would be shrunk or expanded but only in the same scale as the overall depth change due to the revised optical path length throughout. The fact that the shapes change dramatically locally confirms that something beyond just index change is responsible for the signal. This index mechanism may also be operating but it is not the dominant influence.

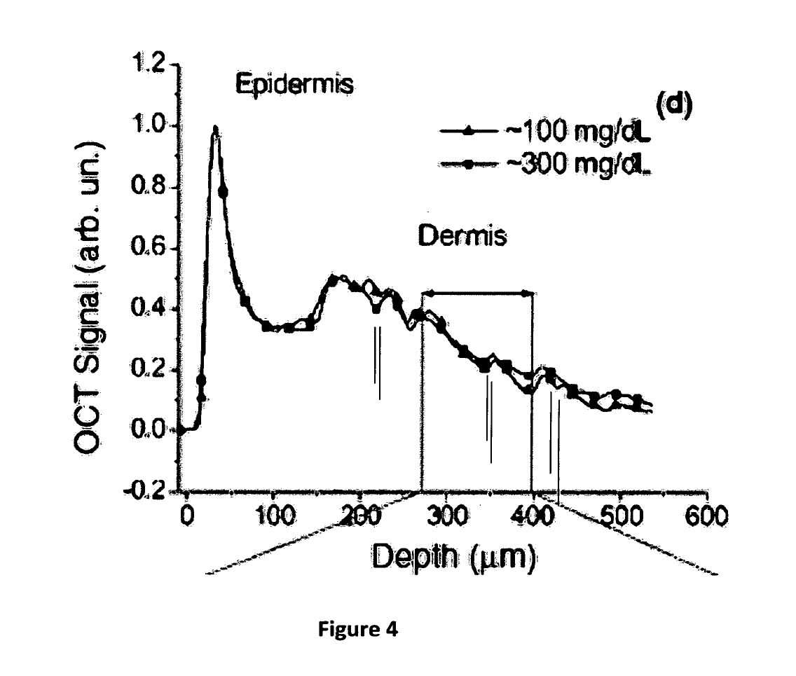

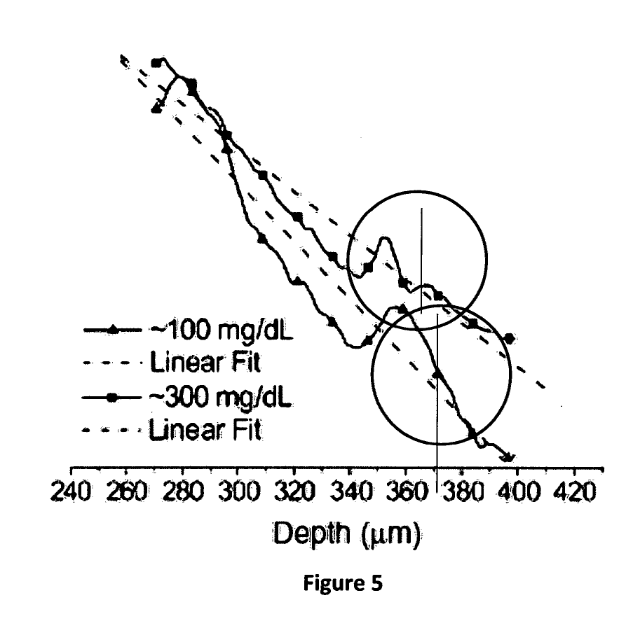

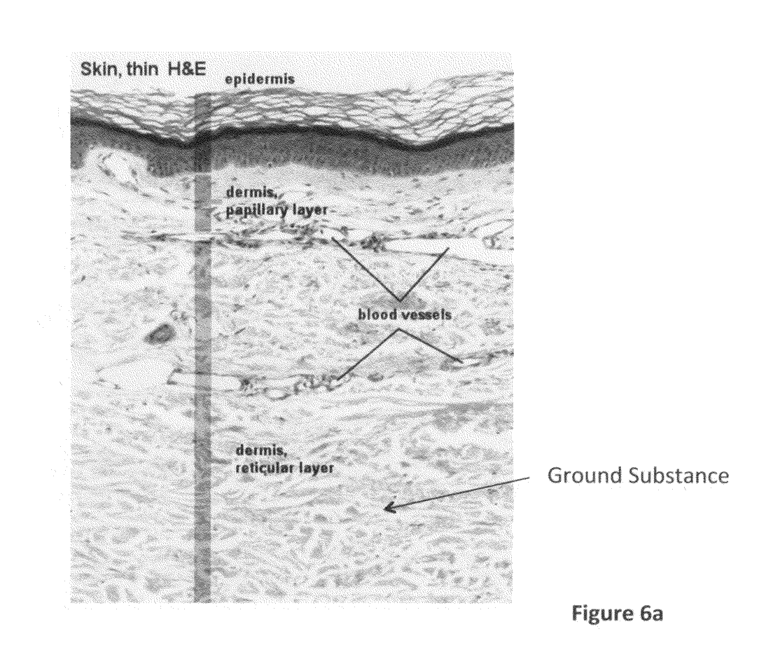

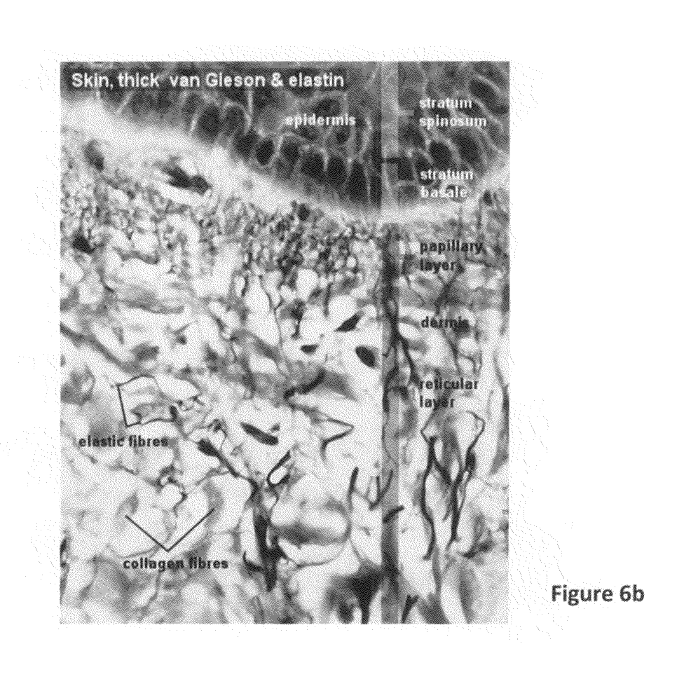

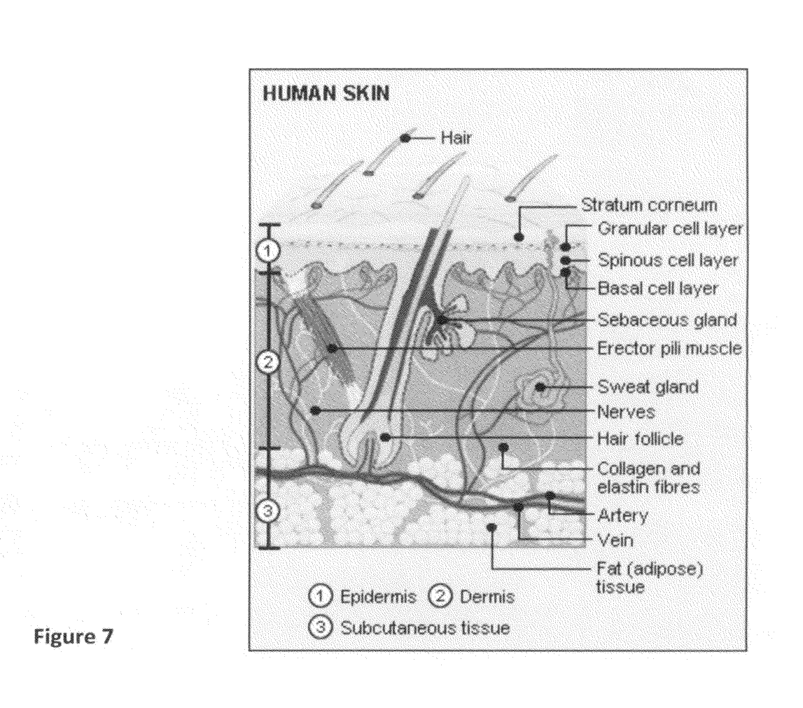

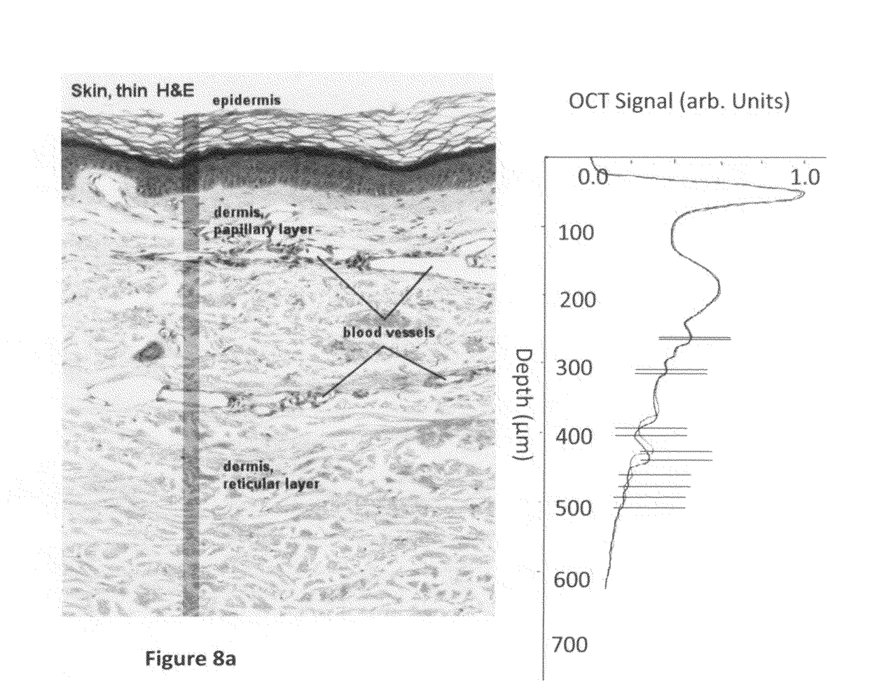

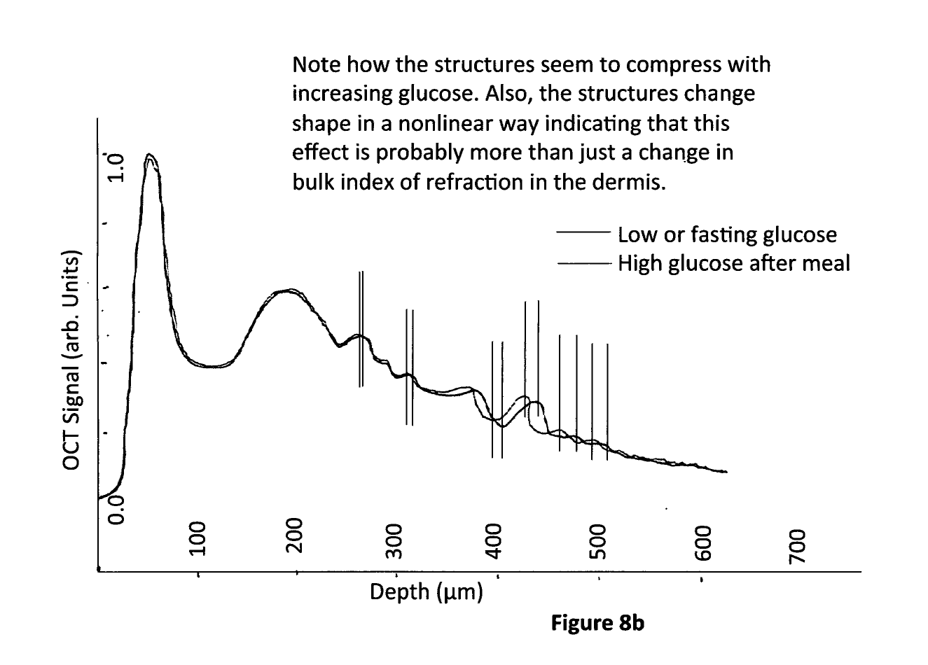

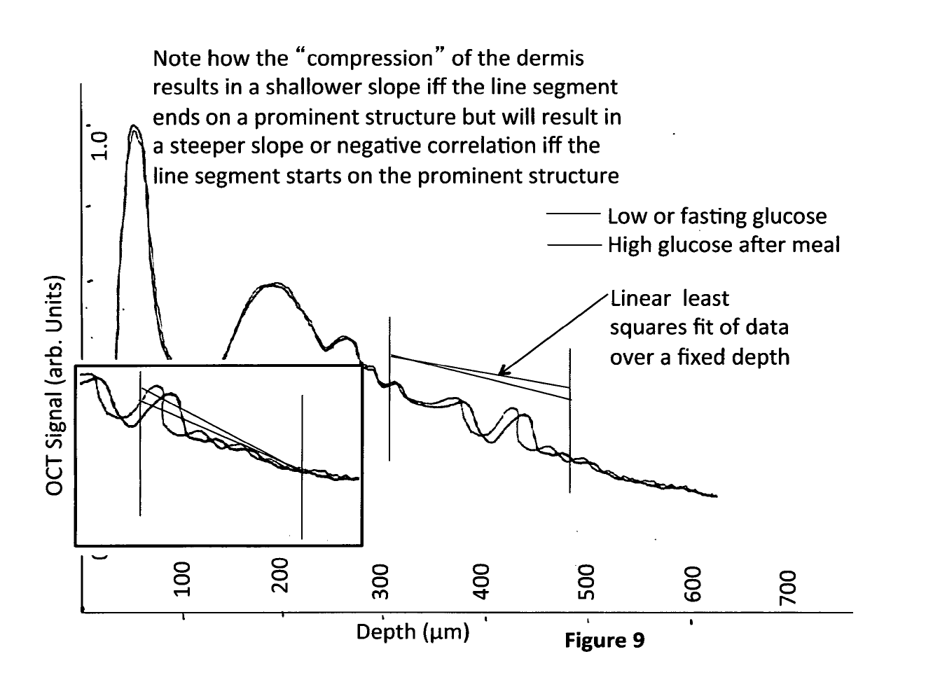

FIG. 4 is a flow chart illustrating the method according to a preferred embodiment. The method according to the invention is comprised of the steps of:

acquiring a first OCT scan S1 of a target at a first time T1; 401

acquiring a second OCT scan S2 of said target at a second time T2; 403

selecting features for analysis in said first scan S1; 405

identifying corresponding features in said second scan S2; 407
analyzing the differences between said selected features; 409
interpreting the results of step 409 with respect to an analyte of interest; 411 and
outputting results 413 of step 411.

In the preferred embodiment, T1 and T2 are times bearing a relation to different levels of glucose in human tissue, such as fasting glucose level and peak glucose level. The features selected in step 405 likewise are selected using criteria related to empirically determined factors relevant to changes in glucose concentration, such as tissue depth. It can be appreciated that calibration is done. To calibrate, what is needed is at least two blood glucose measurements separated by a glucose (or other analyte of interest) change that is greater than the minimum resolution of the OCT or other measuring instrument. In general, the larger the separation of the two glucose/analyte of interest levels, the better the accuracy of the calibration.

Output of the method, step 413, may take the form of a numerical quantification of glucose concentration change, or some other graphic or number based indicia expressing the results of the analysis according to the inventive method. Other forms of display of results, including graphs and multi dimensional projection displays can easily be conceived, and are considered within the scope of the invention taught herein.

It can be appreciated by those of skill in the relevant arts that tracking features in a target of interest is not limited to glucose as an analyte of interest. However, for the purposes of the preferred embodiment, OCT scans of human tissue demonstrate a measurable effect that reproducibly corresponds to changes in glucose concentration. The processing of the OCT scan data may or may not be performed by a CPU that may be associated with the scanning device itself.

Supplemental Material Useful in Understanding the Invention: Appendix A

What is claimed is:

1. A method of determining a characteristic of interest of an analyte of interest by means of OCT scan results of a target of interest, said method comprising the steps of:
obtaining a first scan and a second scan of a target of interest;
identifying at least one scan feature corresponding to target features in said first scan and in said second scan;
comparing the position of said feature in said first scan and the position of said feature in said second scan;
determining, from said comparison of position of said feature in said first scan and said second scan, changes in said analyte of interest in said target of interest; and
outputting results.

2. The method as in claim 1 wherein the step of obtaining a first scan and a second scan include the substep of preparing said target of interest such that said first scan is obtained at first glucose level and said second scan is obtained at a second glucose level.

3. The method as in claim 1 wherein the step of comparing the position includes comparing any of a plurality of topographical changes, including changes in feature shape, feature position relative to other target structures, and feature depth, and where the magnitude of said change in position may be a function of depth.

4. The method as in claim 3 wherein the step of comparing the position further includes comparing any of a plurality of topographical changes and where said magnitude of said change in position may be a function of depth, wherein said function of depth is approximately a linear function.

5. The method as in claim 1 wherein the step of identifying at least one scan feature corresponding to target features includes identifying any of an aggregate of features which may collectively correspond to a trackable target feature, where said trackable target feature may be any of some predetermined feature of interest, including, for example, a set of structures, a structure shape, a micro topography with respect to structure orientation or depth.

6. The method as in claim 1 wherein said feature of interest is related to changes in the thickness of dermis layers.

7. The method as in claim 6 wherein said changes in the thickness of the dermis layer is attributable in some part to changes in hyaluronic acid in the dermis layer.

8. A method of quantifying changes in concentration of glucose in tissue by means of statistically correlating tissue thickness variation to hyaluronic acid level changes, said method comprising the steps of:
obtaining, using an OCT system, a first scan and a second scan of a target of interest
identifying a scan feature in said first scan and identifying said feature in said second scan
comparing the position of said feature in said first scan and the position of feature in said second scan
determining, from said comparison of position of said feature in said first scan and said second scan, a change in analyte of interest in said target of interest.

9. The method as in claim 8 wherein said first scan is obtained at first glucose level and said second scan is obtained at a second glucose level.

10. The method as in claim 8 wherein the magnitude of said change in position is a function of depth.

11. The method as in claim 10 wherein said function of depth is approximately a linear function.

12. The method as in claim 8 wherein the step of identifying at least one scan feature corresponding to target features includes identifying any of an aggregate of features which may collectively correspond to a trackable target feature, where said trackable target feature may be any of some predetermined feature of interest, including, for example, a set of structures, a structure shape, a micro topography with respect to structure orientation or depth.

13. The method as in claim 8 wherein said feature of interest is related to changes in the thickness of dermis layers.

14. The method as in claim 12 wherein said changes in the thickness of the dermis layer is attributable in some part to changes in hyaluronic acid in the dermis layer.
